(19) United States
(12) Patent Application Publication
(10) Pub. No.: US 2021/0299017 A1
(43) Pub. Date: Sep. 30, 2021

(54) ANTIMICROBIAL COMPOSITION

(71) Applicant: Covalon Technologies Inc., Mississauga (CA)

(72) Inventors: Valerio DiTizio, Mississauga (CA); Brian Pedlar, Mississauga (CA); Agnesa Shala-Lawrence, Mississauga (CA); Vyacheslav Dudnyk, Mississauga (CA); Genevieve Conant, Mississauga (CA)

(21) Appl. No.: 17/213,783

(22) Filed: Mar. 26, 2021

Related U.S. Application Data
(60) Provisional application No. 63/000,810, filed on Mar. 27, 2020.
(60) Provisional application No. 63/007,466, filed on Apr. 9, 2020.

(51) Int. Cl. A61K 8/41; A61Q 17/00; A61L 2/18

(52) U.S. Cl. CPC A61K 8/416; A61Q 17/005; A61L 2202/24; A61K 2800/30; A61L 2202/26; A61L 2/18

(57) ABSTRACT

An antimicrobial composition is described herein. The composition comprises an antimicrobial agent, a sequestering agent, a release agent, and an optional dispersing agent. Hand sanitizers and sprays comprising the composition are also described, along with related methods of sanitizing items, such as hands, surfaces, and personal protective equipment.

### RELATED APPLICATIONS

The present application claims the benefit of U.S. Provisional Application Ser. No. 63/000,810, filed Mar. 27, 2020, and U.S. Provisional Application Ser. No. 63/007,466, filed Apr. 9, 2020, the disclosure of each of which is hereby incorporated by reference in its entirety.

### FIELD

The present invention relates to antimicrobial compositions. More specifically, the present invention is, in aspects, concerned with antimicrobial compositions, sprays, sanitizers, and related methods of making and using same.

### BACKGROUND

Antimicrobial compositions for sanitizing surfaces are known in the art. Most hand sanitizers, for example, contain alcohol as their main active ingredient and, with high enough concentrations, are effective at killing viruses like SARS-CoV-2, bacteria and other pathogens. Unfortunately, not only does the alcohol contribute to increased dryness and/or aging of the skin when used repeatedly and for long periods of time, but once the alcohol has evaporated, the sanitizer stops killing microbes.

There is a need for alternative compositions to overcome or mitigate at least some of the deficiencies of the prior art, or to provide a useful alternative.

### SUMMARY

In accordance with an aspect, there is provided an antimicrobial composition comprising an antimicrobial agent, a sequestering agent, and a release agent.

In an aspect, the antimicrobial agent interacts with lipoidal structures, such as lipid bilayers or micelles.

In an aspect, the antimicrobial agent comprises a quaternary ammonium compound.

In an aspect, the antimicrobial agent comprises benzalkonium chloride, benzethonium chloride, methylbenzethonium chloride, cetalkonium chloride, cetylpyridinium chloride, cetrimonium, cetrimide, dofanium chloride, tetraethylammonium bromide, didecyldimethylammonium chloride and domiphen bromide, hexadecyltrimethylammonium bromide, dimethyloctadecyl [3-(trimethoxysilyl) propyl] ammonium chloride or combinations thereof.

In an aspect, the antimicrobial agent comprises benzalkonium chloride.

In an aspect, the antimicrobial agent is present in the composition in an amount of from about 0.01% to about 5% w/v.

In an aspect, the sequestering agent comprises a neutral lipid capable of forming a bilayer.

In an aspect, the sequestering agent comprises a phospholipid.

In an aspect, the sequestering agent comprises a diacylglyceride, such as phosphatidylethanolamine, or phosphatidylcholine, sphingomyelin, or a neutral glycosphingolipid, or combinations thereof.

In an aspect, the sequestering agent comprises phosphatidylcholine.

In an aspect, the sequestering agent is present in the composition in an amount of from about 0.05% to about 10% w/v.

In an aspect, the release agent comprises a surfactant.

In an aspect, the release agent comprises a non-ionic surfactant, a cationic surfactant, or a combination thereof.

In an aspect, the non-ionic surfactant comprises a fatty alcohol.

In an aspect, the fatty alcohol comprises myristyl alcohol, lauryl alcohol, cetyl alcohol, stearyl alcohol, or combinations thereof.

In an aspect, the fatty alcohol comprises myristyl alcohol.

In an aspect, the cationic surfactant comprises a double-chain cationic surfactant.

In an aspect, the double chain cationic surfactant comprises didodecyldimethylammonium bromide (DDAB), didecyldimethylammonium bromide, dioctadecyldimethylammonium bromide, ethonium, or combinations thereof.

In an aspect, the release agent is present in the composition in an amount of from about 0.001% to about 5% w/v, such as from about 0.01% to about 5% w/v.

In an aspect, the composition is alcohol free.

In an aspect, the composition does not comprise an alcohol.

In an aspect, the composition further comprises a dispersing agent.

In an aspect, the dispersing agent comprises an alcohol.

In an aspect, the alcohol comprises ethanol, isopropyl alcohol, or a combination thereof.

In an aspect, the alcohol comprises ethanol.

In an aspect, the dispersing agent is present in the composition in an amount of from about 1% to about 95% w/v.

In an aspect, the composition has an immediate antimicrobial effect.

In an aspect, the composition has a sustained antimicrobial effect.

In an aspect, the antimicrobial effect is sustained from about 1 hour to about 7 days following application.

In an aspect, the antimicrobial effect is sustained for greater than 7 days following application.

In an aspect, the antimicrobial effect is sustained for up to about 30 days following application.

In an aspect, the composition is effective against bacteria, viruses, fungi, yeast, or combinations thereof.

In an aspect, the composition is effective against pathogens comprising a lipid envelope.

In an aspect, the composition is effective against pathogens comprising a net negative charge.

In an aspect, the composition sequesters microbes.

In an aspect, the composition forms an antimicrobial barrier on a surface to which it is applied.

In an aspect, the composition provides a water barrier function when applied to skin or other surfaces.

In an aspect, the composition provides an emollient function when applied to skin or other surfaces.

In an aspect, the composition is effective against pathogens such as MRSA, E. coli, coronaviruses such as SARS-CoV-1, SARS-CoV-2, and MERS-CoV; influenza viruses such as H1N1, H5N1; Acinetobacter baumannii, Escherichia coli, Haemophilus influenzae, Klebsiella pneumoniae, Candida albicans, or combinations thereof.

In accordance with an aspect, there is provided a hand sanitizer comprising the composition described herein.

In accordance with an aspect, there is provided a spray comprising the composition described herein.

In accordance with an aspect, there is provided personal protective equipment (PPE) comprising the composition described herein.

In accordance with an aspect, there is provided a method of sanitizing PPE, the method comprising applying the composition described herein to the PPE.

In an aspect, the PPE comprises a face mask, such as an N95 face mask, vision protection, gloves, a gown, or a combination thereof.

In accordance with an aspect, there is provided a method of sanitizing a surface, the method comprising applying the composition described herein to the surface.

In an aspect, the surface comprises skin, furniture, medical equipment, fabric, textiles, paper, kitchen surfaces, or combinations thereof.

Other features and advantages of the present invention will become apparent from the following detailed description. It should be understood, however, that the detailed description and the specific examples while indicating embodiments of the invention are given by way of illustration only, since various changes and modifications within the spirit and scope of the invention will become apparent to those skilled in the art from said detailed description.

### DETAILED DESCRIPTION

Described herein, in aspects, are antimicrobial compositions that are designed to kill viruses, bacteria and other pathogens on contact, as well as trap and kill these pathogens for as little as about 24 hours to up to several weeks, when applied to surfaces, including skin. The compositions have been specifically formulated to be effective at trapping, deactivating and killing pathogens such as SARS-CoV-2 not only on contact, but also providing persistent protection over time. Also described are mask sanitizers that can be sprayed on a surgical or N95 mask prior to use, or after use if the mask is intended to be reused. The mask sanitizer ensures rapid killing of bacteria, fungi, and viruses on contact, and also maintains persistent antimicrobial protection for up to about 24 hours as the surface of the mask comes into contact with viruses like SARS-CoV-2, bacteria or other pathogens.

### Definitions

The term “microbe(s)” includes, but is not limited to, bacteria, fungi, protozoa, yeast, and viruses.

Viruses include, but are not limited to, rhinovirus, echovirus, rotavirus, respiratory syncytial virus, papilloma virus, papova virus, cytomegalovirus, echinovirus, arbovirus, huntavirus, coxsackie virus, mumps virus, measles virus, rubella virus, polio virus, small pox, Epstein Barr virus, human immunodeficiency virus type I (HIV-1), human immunodeficiency virus type II (HIV-II), hepatitis type A virus, hepatitis type B virus, hepatitis type C virus, influenza viruses such influenza A virus (e.g. sub-type H1N1 or H5N1), influenza B virus, herpes simplex type I (HSV-I) virus, and herpes simplex type II (HSV-II) virus and coronaviruses such as SARS-CoV-1, SARS-CoV-2, and MERS-CoV; influenza viruses such as.

Bacteria include, but are not limited to, Escherichia coli, Acinetobacter baumannii, Haemophilus influenzae, Klebsiella pneumoniae, Staphylococcus aureus, Enterococcus faecials, Proteus vulgaris, Staphylococcus viridans, and Pseudomonas aeruginosa, mycobacteria Rickettsia, Mycoplasma, Neisseria, S. pneumonia, Borrelia burgdorferi, Bacillus antracis, Streptococcus, Staphylococcus, Mycobacterium, Pertissus, Vibrio cholerae, Yersinia pestis, Corynebacterium diphtheriae, Chlamydia trachomatis, and Legionella.

Protozoa include, but are not limited to, Leishmania, Kokzidioa, or Trypanosoma schistosoma.

Fungi include, but are not limited to, Ascomycetes such as Ophiostoma spp., Ceratocystis spp., Aureobasidium pullulans, Sclerophoma spp., Caetomium spp., Humicola spp., Petriella spp., Trichurus spp.; Basidiomycetes such as Coniophora spp., Coriolus spp., Gloeophyllum spp., Lentinus spp., Pleurotus spp., Poria spp., Serpula spp. and Tyromyces spp., Deuteromycetes such as Aspergillus spp., Cladosporium spp., Penicillium spp., Trichoderma spp., Alternaria spp., Paecilomyces spp. and Zygomycetes such as Mucor spp., Candida spp., such as Candida albicans, and Saccharomyces cerevisae.

In understanding the scope of the present application, the articles “a”, “an”, “the”, and “said” are intended to mean that there are one or more of the elements. Additionally, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives.

It will be understood that any aspects described as “comprising” certain components may also “consist of” or “consist essentially of,” (or vice versa) wherein “consisting of” has a closed-ended or restrictive meaning and “consisting essentially of” means including the components specified but excluding other components except for materials present as impurities, unavoidable materials present as a result of processes used to provide the components, and components added for a purpose other than achieving the technical effects described herein. For example, a composition defined using the phrase “consisting essentially of” encompasses any known pharmaceutically acceptable additive, excipient, diluent, carrier, and the like. Typically, a composition consisting essentially of a set of components will comprise less than 5% by weight, typically less than 3% by weight, more typically less than 1% by weight of non-specified components.

It will be understood that any component defined herein as being included may be explicitly excluded by way of proviso or negative limitation, such as any specific compounds or method steps, whether implicitly or explicitly defined herein.

In addition, all ranges given herein include the end of the ranges and also any intermediate range points, whether explicitly stated or not.

Finally, terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

### Compositions

The compositions described herein have, in aspects, three mechanisms that provide antimicrobial protection: (1) rapid killing of bacteria, fungi, yeast, and viruses on contact, (2) maintains persistent antimicrobial protection for from about 24 hours to up to several weeks due to its non-volatile active agent and proprietary delivery system, and (3) traps pathogens that contact its persistent barrier to increase kill rates and reduce the spread of the pathogens.

Most hand sanitizers contain alcohol as their main active ingredient and, with high enough concentrations, are effective at killing viruses like SARS-CoV-2, bacteria and other pathogens. Not only does the presence of the alcohol cause drying out of and/or aging of the skin with repeated use, but once the alcohol has evaporated, the sanitizer stops killing microbes.

The compositions described herein are in aspects designed to provide the same contact-killing effectiveness of high concentration alcohol-based sanitizers, albeit in aspects, comprising little or no alcohol in the composition. In addition, the composition also remains active on the skin or surface it was applied to and continues to trap and kill viruses, bacteria and other pathogens for from about 24 hours to about several weeks. The unique antimicrobial composition described herein provides rapid killing of bacteria, fungi, yeast, and viruses on contact, and has been shown to be very effective against a wide range of microbes, such as Gram-negative and Gram-positive bacteria, as well as fungi and viruses, especially enveloped viruses like SARS-CoV-2.

In aspects, the composition described herein forms an invisible barrier that performs two key functions. The barrier is typically comprised of non-volatile lipid ingredients that keep the antimicrobial ingredient benzalkonium chloride (BAC) active for from about 24 hours to up to about several weeks. The barrier also attracts and traps pathogens that come into contact with the barrier, which enhances exposure of the pathogen to the antimicrobial agent.

The compositions described herein typically use an alcohol such as ethanol as dispersing agent, which combined with an antimicrobial agent, begins killing pathogens on contact. For example, the non-volatile lipid components are dissolved in a 40% ethanol solution, which allows for easy dispersal and rapid drying when applied to any surface, while simultaneously providing an additional contact killing effect that ethanol is well known for. In other aspects, the alcohol is absent or used at a lower level, such as about 1%. Once the ethanol has evaporated, a protective lipid film remains, for example, for up to about 24 hours or, for example, up to about a week, or for example, up to about several weeks, and is typically composed of the active agent BAC, phosphatidylcholine (PC), myristyl alcohol, and a cationic surfactant, such as ethonium and/or didodecyldimethylammonium bromide (DDAB). PC forms the majority of the lipid barrier in which the other components are embedded within. DDAB endows the lipid film with a positive charge, fulfilling two protective functions. Most microbes and enveloped viruses possess a net negative charge, which means they will be attracted to and eventually bind to a lipid film that possesses a net positive charge such as that created by the compositions described herein. In addition, DDAB helps to enhance the activity of BAC by interacting with any anionic contaminants that would otherwise prematurely deactivate BAC, which also possesses a positive charge. Finally, the fatty alcohol (myristyl alcohol) helps to stabilize the other lipid components of the compositions described herein while also providing an emollient effect when applied to skin.

One of the main components in the skin's natural barrier to pathogens are lipids. Lipids act as a natural emollient to soften skin and are key compounds in the skin's natural defense against viruses. The protective barrier that the compositions described herein create on the skin mimics the natural choline-containing fatty acids that are found on skin. The difference is that the lipid barrier provided by the composition also contains BAC that actively kills pathogens, including enveloped viruses like SARS-CoV-2. The water-repellent nature of the lipid barrier also helps to prolong it effectiveness as it will not be easily removed by exposure to moisture.

It will be understood that the active ingredients in the compositions described herein have a long history of being highly effective on enveloped viruses including coronaviruses. Alcohol is widely accepted by all health agencies as an effective antimicrobial agent for killing SARS-CoV-2 and other viruses. BAC is highly effective at deactivating and killing enveloped viruses like SARS-CoV-2 and has been extensively tested on coronaviruses such as SARS-CoV-1 and MERS-CoV as well as similar flu viruses including Influenza, H1N1 and H5N1. BAC also has been shown to have potent antibacterial and antifungal activity against such pathogens as Acinetobacter baumannii, Escherichia coli, Haemophilus influenzae, Klebsiella pneumoniae, drug-resistant Staphylococcus aureus, and Candida albicans amongst many others.

Thus, described herein is an antimicrobial composition comprising an antimicrobial agent, a sequestering agent, a release agent, and an optional dispersing agent.

In typical aspects, the antimicrobial agent interacts with lipoidal structures, such as lipid bilayers or micelles, for example. Typically, the antimicrobial agent is a quaternary ammonium compound, such as benzalkonium chloride, benzethonium chloride, methylbenzethonium chloride, cetalkonium chloride, cetylpyridinium chloride, cetrimonium, cetrimide, dofanium chloride, tetraethylammonium bromide, didecyldimethylammonium chloride and domiphen bromide, hexadecyltrimethylammonium bromide, dimethyloctadecyl [3-(trimethoxysilyl) propyl] ammonium chloride or combinations thereof. Typically, the antimicrobial agent is benzalkonium chloride (BAC).

In aspects, the antimicrobial agent is an antibacterial agent. Some non-limiting exemplary antibacterial agents include those classified as aminoglycosides, beta lactams, quinolones or fluoroquinolones, macrolides, sulfonamides, sulfamethaxozoles, tetracyclines, streptogramins, oxazolidinones (such as linezolid), clindamycins, lincomycins, rifamycins, glycopeptides, polymxins, lipo-peptide antibiotics, metal salts, as well as pharmacologically acceptable sodium salts, pharmacologically acceptable calcium salts, pharmacologically acceptable potassium salts, lipid formulations, derivatives and/or analogs of the above.

In other aspects, the antimicrobial agent includes an antifungal agent. Some exemplary classes of antifungal agents include imidazoles or triazoles such as clotrimazole, miconazole, ketoconazole, econazole, butoconazole, omoconazole, oxiconazole, terconazole, itraconazole, fluconazole, voriconazole (UK 109,496), posaconazole, ravuconazole or flutrimazole; the polyene antifungals such as amphotericin B, liposomal amphoterecin B, natamycin, nystatin and nystatin lipid formulations; the cell wall active cyclic lipopeptide antifungals, including the echinocandins such as caspofungin, micafungin, anidulfungin, cilofungin; LY121019; LY303366; the allylamine group of antifungals such as terbinafine. Yet other non-limiting examples of antifungal agents include naftifine, tolnaftate, mediocidin, candicidin, trichomycin, hamycin, aurefungin, ascosin, ayfattin, azacolutin, trichomycin, levorin, heptamycin, candimycin, griseofulvin, BF-796, MTCH 24, BTG-137586, pradimicins (MNS 18184), benanomicin; ambisome; nikkomycin Z; flucytosine, or perimycin.

In still other aspects, the antimicrobial includes an antiviral agent. Non-limiting examples of antiviral agents include cidofovir, amantadine, rimantadine, acyclovir, gancyclovir, pencyclovir, famciclovir, foscarnet, ribavirin, or valacyclovir. In some aspects the antimicrobial agent is an innate immune peptide or protein. Some exemplary classes of innate peptides or proteins are transferrins, lactoferrins, defensins, phospholipases, lysozyme, cathelicidins, serprocidins, bacteriocidal permeability increasing proteins, amphipathic alpha helical peptides, and other synthetic antimicrobial amino acids, peptides, or proteins.

In typical aspects, the antimicrobial agent may comprise chlorhexidine, octenidine, benzalkonium chloride, benzethonium chloride, polyhexamethylene biguanide, copper, zinc, silver, chlorine, fluoroquinolones, b-lactams, macrolides, aminoglycosides, tetracyclines, or combinations thereof.

Typically, the antimicrobial agent is present in the composition in an amount of from about 0.01% to about 5% w/v, such as from about 0.01%, about 0.05%, about 0.1%, about 0.5%, about 1%, about 1.5%, about 2%, about 2.5%, about 3%, about 3.5%, about 4%, or about 4.5% to about 0.05%, about 0.1%, about 0.5%, about 1%, about 1.5%, about 2%, about 2.5%, about 3%, about 3.5%, about 4%, about 4.5%, or about 5% w/v. For example, about 0.1%, about 0.15%, or about 0.2% w/v. Typically, about 0.13% w/v.

The sequestering agent in the composition is typically an agent capable of slowing release of the antimicrobial agent. In typical aspects, the sequestering agent comprises a neutral lipid capable of forming a bilayer. For example, in typical aspects, the sequestering agent comprises an amphipathic compound, such as a phospholipid. Examples of phospholipids include phosphosphingolipids, such as ceramide phosphorylcholine, ceramide phosphorylethanolamine, and ceramide phosphoryllipiddiacylglycerides, and diacylglycerides, such as phosphatidic acid, phosphatidylethanolamine, phosphatidylcholine, phosphatidylserine, phosphatidylinositol, phosphatidylinositol phosphate, phosphatidylinositol bisphosphate, and phosphatidylinositol trisphosphate. Combinations of sequestering agents or phospholipids are contemplated. Typically, the sequestering agent comprises phosphatidylcholine.

In typical aspects, the sequestering agent is present in the composition in an amount of from about 0.05% to about 10% w/v, such as from about 0.05%, about 0.1%, about 0.5%, about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 9%, or 9.5% to about 0.1%, about 0.5%, about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 9%, 9.5%, or about 10% w/v. For example, about 0.5% w/v.

In typical aspects, the release agent is a compound that is capable of disrupting the sequestering agent in order to slowly release the antimicrobial agent over a period of time. For example, the release agent is typically a surfactant, such as a non-ionic surfactant, a cationic surfactant, or a combination thereof. Examples of non-ionic surfactants include polyglycerol alkyl ethers, glucosyl dialkyl ethers, crown ethers, ester-linked surfactants, polyoxyethylene alkyl ethers, Brij, Spans (sorbitan esters) Tweens (Polysorbates), ethoxylates, fatty acid esters of polyhydroxy compounds, amine oxides, fatty alcohols, and combinations thereof. Typically, the non-ionic surfactant comprises a fatty alcohol, such as myristyl alcohol, lauryl alcohol, cetyl alcohol, stearyl alcohol, or combinations thereof. Typically, the fatty alcohol comprises myristyl alcohol.

Examples of cationic surfactants include double-chain cationic surfactants such as DDAB, didecyldimethylammonium bromide, dioctadecyldimethylammonium bromide, ethonium, or combinations thereof.

In typical aspects, a non-ionic surfactant, such as myristyal alcohol, and a cationic surfactant, such as DDAB and/or ethonium, are used in combination as the release agent. When two or more surfactants are used in combination in the compositions described herein, they may be used in various ratios. For example, when two surfactants are used, they may be used in a ratio of, for example, from about 0.01:0.99 to about 0.99:0.01 (w/v), such as from about 0.10:0.90 to about 0.90:0.10 (w/v); from about 0.20:0.80 to about 0.80:0.20 (w/v);from about 0.3:0.7 to about 0.7:0.3 (w/v); or from about 0.6:0.4 to about 0.4:0.6 (w/v) based on the total mass of surfactant added.

The release agent is typically present in the composition in an amount of from about 0.001% to about 5% w/v, such as from about 0.001%, about 0.005%, about 0.01%, about 0.05%, about 0.1%, about 0.5%, about 1%, about 1.5%, about 2%, about 2.5%, about 3%, about 3.5%, about 4%, or about 4.5% to about 0.005%, about 0.01%, 0.05%, about 0.1%, about 0.5%, about 1%, about 1.5%, about 2%, about 2.5%, about 3%, about 3.5%, about 4%, about 4.5%, or about 5% w/v. For example, about 0.01%, about 0.05%, or about 0.1% w/v, such as from about 0.01% to about 5% w/v.

The composition described herein usually comprises a dispersing agent, which typically comprises an alcohol, such as ethanol, isopropyl alcohol, or a combination thereof. Typically, the alcohol comprises ethanol. The alcohol is typically present in the composition in an amount of from about 1% to about 95% w/v, such as from about 1%, about 2%, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, or about 90% to about 2%, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95% w/v. In aspects, when the composition comprises the alcohol, the alcohol is present in the composition in a low amount, such as in an amount from about 0.1% to about 4% w/v, and most typically, in an amount of about 2% w/v. Alcohol may also be absent from the composition in certain aspects, such that the composition is considered an “alcohol free” composition. In these aspects, the composition does not comprises an alcohol.

The composition described herein typically has an immediate antimicrobial effect, meaning that the dispersing agent and/or antimicrobial agent is able to kill or inactivate microbes upon contact. In additional or alternative aspects, the composition described herein has a sustained antimicrobial effect, meaning that the composition is able to trap and/or kill/inactivate microbes over a period of time, ranging from minutes to days. For example, in typical aspects, the antimicrobial effect is sustained from about 1 hour to about 30 days following application, such as from about 1 hour, about 2 hours, about 3 hours, about 6 hours, about 12 hours, about 18 hours, about 24 hours, about 36 hours, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 26 days, about 27 days, about 28 days, or about 29 days to about 2 hours, about 3 hours, about 6 hours, about 12 hours, about 18 hours, about 24 hours, about 36 hours, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 26 days, about 27 days, about 28 days, about 29 days or about 30 days.

In aspects, the antimicrobial effect is sustained from about 1 hour to about 7 days following application. In other aspects, the antimicrobial effect is sustained for greater than 7 days following application. In further aspects, the antimicrobial effect is sustained for up to about 30 days following application.

It will be understood that the composition can be designed to be effective against bacteria, viruses, fungi, yeast, or combinations thereof, depending upon the antimicrobial agent chosen for use. Typically, the composition is effective against pathogens comprising a lipid envelope and/or pathogens comprising a net negative charge. Typically, the composition described herein is effective against pathogens such as MRSA, E. coli, coronaviruses such as SARS-CoV-2, SARS-CoV-1, and MERS-CoV; influenza viruses such as H1N1, H5N1; Acinetobacter baumannii, Escherichia coli, Haemophilus influenzae, Klebsiella pneumoniae, Candida albicans, or combinations thereof.

Typically, the composition sequesters or traps microbes and forms an antimicrobial barrier on a surface to which it is applied. In additional or alternative aspects, the composition provides a water barrier function when applied to skin or other surfaces and/or an emollient function when applied to skin or other surfaces.

The compositions may comprise additional agents and/or excipients as will be understood to a skilled person. For example, water hardness controlling excipients such as disodium EDTA may be included, pH adjusting excipients or buffers such as sodium bicarbonate may be included, and/or fragrances such as essential oils, including but not limited to, those extracted from anise, orange, basil, birch, cinnamon (e.g., cinnamon zeylanicum), cedarwood, cyprus, citronella, eucalyptus (e.g., eucalyptus globulus), incense, lavender, hybrid lavender, frankincense, fennel, lemongrass, lemon, tangerine, mint, spearmint, myrrh, oregano, peppermint, grapefruit, mountain savoury, rose, rosemary, rose geranium, sage, sandalwood, thyme, ginger, mustard or combinations thereof, may be included in the compositions described herein. The essential oil, when present, is typically present in the composition in an amount of from about 0.01% to about 3% w/v, such as from about 0.01%, about 0.02%, about 0.03%, about 0.04%, about 0.05%, about 0.06%, about 0.07%, about 0.08%, about 0.09%, about 1%, about 1.5%, about 2%, or about 2.5% to about 0.02%, about 0.03%, about 0.04%, about 0.05%, about 0.06%, about 0.07%, about 0.08%, about 0.09%, about 1%, about 1.5%, about 2%, about 2.5% or about 3%. Typically, the essential oil is present in an amount of about 0.2% w/v.

Also described herein are hand sanitizers, surface sprays, and compositions for application to the human body comprising the composition. For example, the composition described herein is suitable as an effective hand sanitizer that has a sustained effect that lasts beyond evaporation of the solvent/dispersing agent. Likewise, as a surface spray, the composition effectively sanitizes porous or non-porous surfaces, providing them with a lasting antimicrobial film. The composition can continue to trap and/or kill or inactivate microbes on the surface to which it is applied for a period of time, from minutes to hours or days or weeks. In other aspects, the composition is, for example, in the form of a gel, cream, ointment, paste, lotion, or spreadable solid, such as a deodorant stick.

The composition described herein is particularly suited to use in sanitizing personal protective equipment (PPE), such as N95 or surgical masks. The masks can be sprayed or dipped in the compositions described herein, which sanitizes the mask without comprising its integrity. The mask can then be reused without substantial risk of cross-contamination. Single use masks may also be provided impregnated with the composition described herein, thereby improving their protection and increasing their lifespan. Thus, also provided herein is PPE comprising the composition described herein.

Various methods are also provided herein, such as a method of sanitizing PPE or sanitizing a surface. The method comprises applying the composition described herein to the PPE or surface and allowing the dispersing agent to evaporate. Examples of PPE include a face mask, such as an N95 face mask or a surgical mask, vision protection, gloves, a gown, or a combination thereof. Examples of surfaces that can be sanitized include skin, furniture, medical equipment, fabric, textiles, paper, kitchen surfaces, or combinations thereof.

The above disclosure generally describes the present invention. A more complete understanding can be obtained by reference to the following specific Examples. These Examples are described solely for purposes of illustration and are not intended to limit the scope of the invention. Changes in form and substitution of equivalents are contemplated as circumstances may suggest or render expedient. Although specific terms have been employed herein, such terms are intended in a descriptive sense and not for purposes of limitation.

### EXAMPLES

### Example 1 —Antimicrobial Spray with Benzalkonium Chloride

### Purpose:

To test the antimicrobial efficacy of spray formulations containing benzalkonium chloride (BAC) on methicillin-resistant Staphylococcus aureus (MRSA) (Gram positive bacteria) and Escherichia coli (Gram negative bacteria), using a Zone of Inhibition (ZOI) test method, when sprayed on 1 cm (diameter) disks cut from a face mask.

### Formulations:

The seven formulations tested are listed in Table 1. All percentages are w/v in water.

TABLE 1
Seven formulations tested for their antimicrobial efficacy
via zone of growth inhibition for both MRSA and E. coli
(benzalkonium chloride—BAC, ethanol—EtOH,
phosphatidylcholine—PC, myristyl alcohol—MyrOH, ethonium—Ethon).

| Formulation | | | | | |
|---|---|---|---|---|---|
| # | BAC (%) | EtOH (%) | PC (%) | MyrOH (%) | Ethon (%) |
| 1 | 0.13 | 40 | 0 | 0 | 0 |
| 2 | 0.13 | 40 | 0.5 | 0 | 0 |
| 3 | 0.13 | 40 | 0.5 | 0.01 | 0 |
| 4 | 0.13 | 40 | 0.5 | 0 | 0.1 |
| 5 | 0.13 | 40 | 0.5 | 0.01 | 0.1 |
| 6 | 0.13 | 40 | 0 | 0 | 0.1 |
| 7 | 0.13 | 40 | 0 | 0.05 | 0.1 |

### Antimicrobial Testing:

Kirby-Bauer antimicrobial tests (5 days for MRSA and 3 days for E. coli) were performed using sanitizer formulations (Table 1) to assess the effectiveness of the different formulations on growth inhibition of MRSA and E. coli bacterial strains.

### Preparation of the Surgical Mask Disk

One cm disks were cut from a surgical mask and they were placed on weigh boats with the blue layer (the inner layer that contacts the skin when in use) facing down (different boats were used for different solutions) and the white layer facing up. To ensure sterility of the disks they were placed in a Biosafety cabinet (BSC) with the UV light on for at least 15 minutes. The prepared formulations (from Table 1) were dispensed into 30 mL amber glass bottles. Using the spray bottle, four pulses of each formulation were applied onto the outer surface (the white layer) of the surgical mask disk. The disks were prepared in triplicate for each formulation. The disks with the sprayed formulations applied to them were left to dry in the BSC for a few minutes (2-5 minutes) prior to their transfer, with sterile tweezers, into bacterial cultures prepared in Mueller Hinton Agar (MHA) plates. To prepare negative controls, disks were sprayed with water instead of the sanitizer formulation.

Inoculation of the MHA Plates with MRSA or E. Coli Strains

Microorganism stock vials of both MRSA and E. coli strains were removed from the −20° C. freezer. E. coli and MRSA were scraped and streaked into Trypsin Soy Agar (TSA) plates. The streaked plates with the bacteria were incubated at 37° C. for 24 hours.

To prepare the microorganism inoculum, two streaks of the colonies (from the above prepared plates) were isolated and transferred into a flint glass tube containing 3 mL of 0.85% w/v NaCl solution. To ensure consistent and appropriate dilutions were used, the absorbance at 625 nm for the suspensions was matched (±0.01) to that of the McFarland 0.5 turbidity standard; the same procedure was repeated for both bacterial strains.

Sterile cotton swabs were dipped in the standardized, well-mixed, inoculum and any excess fluid was removed by squeezing the swab against the side of the tube. The inoculum was spread across half of the surface of the square MHA plate, where an additional swab was used to inoculate the remaining half of the plate. MHA plates were used for E. coli, whereas MHA plates supplemented with 4% NaCl were used for MRSA. The plates were allowed to dry for 5 minutes, prior to applying the disk samples prepared as described above. The plates were incubated at 35±2° C. for 16-18 hours.

For the negative control test, disks prepared with water (see above) were transferred into the inoculated plates with the bacteria strains.

### Transfer of the Disks for Continued Zone of Inhibition Testing on Day 2, 3, 4, and 5

In the case where the disks with a sanitizer formulation provided a zone of inhibition, these disks were transferred to newly inoculated plates to monitor and determine the duration of inhibition and, therefore, the sustained efficacy of the sanitizer. Using sterile tweezers, the disks with the white layer facing the inoculated plate were transferred into a new plate, in the same direction, with the white layer facing the newly inoculated plate.

### Results and Discussion:

All the formulations containing BAC (at a concentration of 0.13%w/v) resulted in a zone of inhibition for at least 24 h regardless of other components in the formulation, confirming excellent antimicrobial activity against both bacterial strains. The data are summarized in Tables 2 and 3. In general, the ZOI for MRSA was greater and persisted longer compared to that of E. coli. Note that in all cases there was no inhibition with water, which was used as a negative control (data not shown).

By analyzing the ZOI data from the most sensitive bacteria, MRSA, it is possible to evaluate the contribution of each of the components to the antimicrobial activity by comparing the ZOIs of formulations containing only a subset of the complete formulation (i.e. formulation 5). The effect of PC can be seen by comparing the ZOIs of formulations 1 and 2. The presence of PC appears to attenuate the activity of BAC through relative decreases in ZOI size on day 1 (from 10 mm to 3.7 mm) and duration of ZOI appearance (from 2 days to 1 day). Since the concentration of BAC in each formulation is the same and the conditions of the study would not lead to removal of BAC (e.g. from evaporation), it's reasonable to assume that the presence of PC was rendering a large portion of the BAC present in formulation 2 immobile and trapping it on the sprayed sample. Surprisingly, this sequestering of BAC by PC appeared to be reversed to some extent by the presence of myristyl alcohol (formulation 3) with the most consistent and favorable results (i.e. with respect to ZOI duration) obtained when either ethonium (formulation 4) or both additives were present (formulation 5).

Additional insight into the contribution of myristyl alcohol and ethonium to the total antimicrobial effect can be obtained by comparing those formulations without PC (i.e. formulations 1, 6, and 7). The time dependent ZOI profiles of formulations 1 and 6 were similar with both leading to large ZOI's on day 1 followed by much smaller zones on day 2 suggesting that ethonium does not contribute substantially to the antimicrobial effect of BAC. However, when myristyl alcohol is added, the ZOI from day 2 is increased and the antimicrobial effect is observed for an additional day. Thus, there appears to be additive or synergistic interactions between BAC and myristyl alcohol at a minimum and perhaps between BAC and the two additives together.

TABLE 2
The growth inhibition zones of various BAC-containing
formulations challenged with MRSA.

| | Inhibition Zone (mm) | | | | |
|---|---|---|---|---|---|
| Formulation # | Day 1 | Day 2 | Day 3 | Day 4 | Day 5 |
| 1 | 10 | 1.3 | — | — | — |
| 2 | 3.7 | — | — | — | — |
| 3 | 3 | 1 | 0.3 | — | — |
| 4 | 4.7 | 3 | 1 | 0.5 | — |
| 5 | 5 | 3 | 1 | 0.5 | — |
| 6 | 10 | 1 | — | — | — |
| 7 | 9.7 | 3.7 | 1.7 | — | — |

TABLE 3
The growth inhibition zones of various BAC-containing
formulations challenged with E. coli.

| | Inhibition Zone (mm) | | |
|---|---|---|---|
| Formulation # | Day 1 | Day 2 | Day 3 |
| 1 | 3 | —* | — |
| 2 | 0.3 | — | — |
| 3 | 0.7 | — | — |
| 4 | 1 | —* | — |
| 5 | 1.7 | —* | — |
| 6 | 4.3 | —* | — |
| 7 | 4 | —* | — |

*Contact inhibition

The overall zone of inhibition for E. coli was about 4 times lower when compared to the zone of inhibition with MRSA, using the same formulations. Considering that E. coli are a Gram-negative bacteria, containing an outer membrane (lipopolysaccharide) and a peptidoglycan, whereas MRSA (Gram-positive) contain a single peptidoglycan membrane, E. coli appear more resistant to the sanitizer formulations which target the microbial membrane, thus explaining the possible reason for the lower inhibition. All 7 formulations tested against E. coli provide a visible zone of inhibition for Day 1. On Day 2, formulations 1, 4, 5, 6 and 7 appeared white underneath, compared to the control (water), which turned yellow indicating bacterial culture growth on the surface. This information suggests that there is contact inhibition on Day 2 for E. coli using formulations 1, 4, 5, 6, and 7.

### Conclusion:

Antimicrobial formulations containing BAC in conjunction with PC, myristyl alcohol, and ethonium display enhanced activity relative to BAC-only formulations as demonstrated through extended duration of ZOI production when challenged with MRSA. The major factor involved in the enhanced activity appears to be the creation of a metastable association of a surface with BAC, PC, myristyl alcohol, and ethonium that results in the slow but sustained release of BAC. An additional, but likely less important, contribution to the enhanced antimicrobial effect may be the existence of additive or synergistic interactions between BAC, myristyl alcohol, and ethonium that potentiate the bactericidal activity of BAC.

### Example 2—Low Alcohol and High Alcohol Antimicrobial Sprays

A low alcohol spray was created with the following ingredients:
2% ethanol, 0.13% benzalkonium chloride (BAC), 0.1% phosphatidylcholine, 0.05% didecyldimethylammonium chloride, 0.05% myristyl alcohol, 0.05% orange essential oil, 0.02% sodium bicarbonate, and 0.01% EDTA.

A high alcohol spray was created with the following ingredients:
40% ethanol, 0.13% benzalkonium chloride (BAC), 0.2% phosphatidylcholine, 0.05% didecyldimethylammonium chloride, 0.05% myristyl alcohol, 0.05% orange essential oil, 0.02% sodium bicarbonate, and 0.01% EDTA.

These sprays are being tested in a hospital setting.

### Example 3—Log Reduction Test for Antimicrobial Composition against Staphylococcus Aureus

### Purpose:

The purpose of this study was to assess the antimicrobial properties of the formulations listed in Table 1 on a non-porous surface against a representative Gram-positive bacterium in a log-reduction experiment. This study examined the residual antimicrobial properties of the novel formulations when surfaces were sanitized prior to application of microbes.

### Formulations:

The two formulations tested are listed in Table 4. All percentages are w/v in water.

TABLE 4
Alcohol-free (Formula A) and low-alcohol (Formula B) formulations were
tested for their antimicrobial efficacy via a log reduction assay using
Staphylococcus aureus as the challenge organism (benzalkonium
chloride—BAC, ethanol—EtOH, phosphatidylcholine—PC, myristyl
alcohol—MyrOH, dimethyloctadecyl [3-(trimethoxysilyl) propyl]
ammonium chloride—(DOAC), didodecyldimethylammonium
bromide—DDAB).

| | Formula A | Formula B |
|---|---|---|
| EtOH (%) | — | 2 |
| DOAC (%) | 0.5 | — |
| BAC (%) | 0.1 | 0.13 |
| DDAB (%) | 0.1 | 0.05 |
| PC (%) | 0.2 | 0.2 |
| MyrOH (%) | — | 0.05 |
| EDTA (%) | 0.1 | 0.01 |
| HCO₃⁻ (%) | — | 0.02 |
| Orange Oil (%) | — | 0.05 |

### Experimental Method:

Glass cover slides were saturated with each antimicrobial solution and allowed to sit at ambient conditions for either 1, 7, or 27 days prior to inoculation with S. aureus. The inoculum was dried on the sanitized surfaces for at least 20 minutes before viable bacteria were recovered and plated onto agar. After a 24 hour incubation, colonies were counted.

### Results:

Table 5 summarizes the results of the log reduction assay, which indicate that both antimicrobial formulations continue to elicit a >4-log reduction in viable bacteria up to 27 days after application to a non-porous surface.

TABLE 5
Residual antimicrobial activity of pre-
sanitized surfaces (1, 7, and 27 days).

| | Formula A | | Formula B | |
|---|---|---|---|---|
| | | Log | | Log |
| Sample | Log (CFU) | Reduction | Log (CFU) | Reduction |
| 1 day Control | 6.27 | — | 6.27 | — |
| 1 day Treatment | 0 | 6.27 | 0 | 6.27 |
| 7 day Control | 6.08 | — | 6.08 | — |
| 7 day Treatment | 0 | 6.08 | 0 | 6.08 |
| 27 day Control | 6.21 | — | 6.21 | — |
| 27 day Treatment | 0 | 6.21 | 0 | 6.21 |

Control = no treatment applied to surface, CFU = colony forming units.

### Conclusion:

As shown in the table above, the alcohol-free and low-alcohol antimicrobial compositions demonstrated excellent residual antimicrobial activity against S. aureus.

The above disclosure generally describes the present invention. Although specific terms have been employed herein, such terms are intended in a descriptive sense and not for purposes of limitation.

All publications, patents and patent applications cited above are herein incorporated by reference in their entirety to the same extent as if each individual publication, patent or patent application was specifically and individually indicated to be incorporated by reference in its entirety.

Although preferred embodiments of the invention have been described herein in detail, it will be understood by those skilled in the art that variations may be made thereto without departing from the spirit of the invention or the scope of the appended claims.

1. An antimicrobial composition comprising an antimicrobial agent, a sequestering agent, and a release agent.

2. The composition of claim 1, wherein the antimicrobial agent comprises a quaternary ammonium compound selected from benzalkonium chloride, benzethonium chloride, methylbenzethonium chloride, cetalkonium chloride, cetylpyridinium chloride, cetrimonium, cetrimide, dofanium chloride, tetraethylammonium bromide, didecyldimethylammonium chloride and domiphen bromide, hexadecyltrimethylammonium bromide, dimethyloctadecyl [3-(trimethoxysilyl) propyl] ammonium chloride and combinations thereof.

3. The composition of claim 1, wherein the antimicrobial agent is present in the composition in an amount of from about 0.01% to about 5% w/v.

4. The composition of claim 1, wherein the sequestering agent comprises a phospholipid selected from a diacylglyceride, such as phosphatidylethanolamine, or phosphatidylcholine, sphingomyelin, or a neutral glycosphingolipid, and combinations thereof.

5. The composition of claim 1, wherein the sequestering agent is present in the composition in an amount of from about 0.05% to about 10% w/v.

6. The composition of claim 1, wherein the release agent comprises a non-ionic surfactant, a cationic surfactant, or a combination thereof.

7. The composition of claim 6, wherein the non-ionic surfactant comprises a fatty alcohol selected from myristyl alcohol, lauryl alcohol, cetyl alcohol, stearyl alcohol, and combinations thereof and wherein the cationic surfactant comprises a double-chain cationic surfactant selected from didodecyldimethylammonium bromide (DDAB), didecyldimethylammonium bromide, dioctadecyldimethylammonium bromide, ethonium, and combinations thereof.

8. The composition of claim 1, wherein the release agent is present in the composition in an amount of from about 0.001% to about 5% w/v, such as from about 0.01% to about 5% w/v.

9. The composition of claim 1, wherein the composition is alcohol free.

10. The composition of claim 1, further comprising a dispersing agent.

11. The composition of claim 10, wherein the dispersing agent comprises an alcohol selected from ethanol, isopropyl alcohol, or a combination thereof.

12. The composition of claim 10, wherein the dispersing agent is present in the composition in an amount of from about 1% to about 95% w/v.

13. The composition of claim 1, wherein the composition has an immediate antimicrobial effect.

14. The composition of claim 1, wherein the composition has an antimicrobial effect that is sustained from about 1 hour to up to about 30 days following application.

15. The composition of claim 1, wherein the composition is effective against bacteria, viruses, fungi, protozoa, yeast, or combinations thereof.

16. The composition of claim 15, wherein the composition is effective against pathogens comprising a lipid envelope and/or pathogens comprising a net negative charge.

17. The composition of claim 1, wherein the composition sequesters microbes.

18. The composition of claim 1, wherein the composition forms an antimicrobial barrier on a surface to which it is applied.

19. The composition of claim 1, wherein the composition provides a water barrier function when applied to skin or other surfaces.

20. The composition of claim 1, wherein the composition provides an emollient function when applied to skin or other surfaces.

21. The composition of claim 1, wherein the composition is effective against pathogens such as MRSA, E. coli, coronaviruses such as SARS-CoV-1, SARS-CoV-2, and MERS-CoV;
influenza viruses such as H1N1, H5N1; Acinetobacter baumannii, Escherichia coli, Haemophilus influenzae, Klebsiella pneumoniae, Candida albicans, or combinations thereof.

22. A hand sanitizer or sanitizing spray comprising the composition of claim 1.

23. A method of sanitizing a surface, the method comprising applying the composition of claim 1 to the surface.

24. The method of claim 23, wherein the surface comprises personal protective equipment (PPE) and wherein the PPE comprises a face mask, such as an N95 face mask, vision protection, gloves, a gown, or a combination thereof.

25. The method of claim 23, wherein the surface comprises skin, furniture, medical equipment, fabric, textiles, paper, kitchen surfaces, or combinations thereof.
